(19) United States
(12) Patent Application Publication
(10) Pub. No.: US 2008/0161307 A1
(43) Pub. Date: Jul. 3, 2008

(54) Organic Compounds

(76) Inventors: David Lewis Earnest, Harding Township, NJ (US); Mikhail Rojavin, Southampton, PA (US); Gervais Tougas, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/815,037

(22) PCT Filed: Jan. 27, 2006
(86) PCT No.: PCT/US06/02927

§ 371 Date: Jul. 30, 2007

Related U.S. Application Data
(60) Provisional application No. 60/648,479, filed on Jan. 31, 2005.

(51) Int. Cl. A61K 31/5375; A61K 31/404; A61P 1/04

(52) U.S. Cl. 514/238.2; 514/415

(57) ABSTRACT

A method for the treatment of delayed gastric emptying, in a patient in need of such treatment, which comprises administering an effective amount of a 5-HT4 agonist, e.g. tegaserod or salts or hydrates thereof, to the patient.

This invention relates to a pharmaceutical composition, use, or method in the treatment of PPI induced delayed gastric emptying with 5-hydroxytryptamine receptor type 4 receptor agonists (5-HT4 agonists).

Proton pump inhibitors (PPIs) are the most effective pharmacological agents used to treat symptoms and complications of acid reflux in patients with gastroesophageal reflux diseases (GERD) (Berardi R. R, 2001, Postgrad.Med., 24-35; Katz P. O., Frissora C., 2002, Curr.Gastroenterol. Rep., 4: 459-462 ). While generally safe, PPIs have been reported to be associated with development of delayed gastric emptying (DeVault, K. R., 1996, Am J Gastroenterol, 91, 1869-1870). For example, rabeprazole has been shown to have a dual effect with overall delay of gastric emptying function (Anjiki H, Sanaka M, Kuyama Y. Dual effects of rabeprazole on solid-phase gastric emptying assessed by the 13C-octanoate breath test. Digestion. 2005; 72(2-3):189-94. Epub Sep. 21, 2005). Omeprazole in daily doses of 20 to 40 mg has been shown to significantly delay the rate of gastric emptying in healthy volunteers (Benini L et al., 1996, Dig Dis Sci, 41, 469-474; Parkman H P et al., 1998, Gastroenterol., 34, 671-675; Rasmussen L et al., 1997, Scand J Gastroenterol., 32, 900-905). The magnitude of this reduction ranges from 15% to as much as 40% of the baseline gastric emptying rate (Benini L et al., 1996, Dig Dis Sci, 41, 469-474; Parkman H P et al., 1998, Gastroenterol., 34, 671-675). While this remains a controversial issue, delayed gastric emptying has been associated with the presence of bacterial overgrowth (Stotzer P. O. et al., 1999, Dig Dis Sci., 44, 729-734) and development of dyspeptic symptoms (Buckles D. C. et al, 2004, Am J Med Sci, 327, 1-4; Haag S. et al., 2004, Gut, 53, 1445-1451). Furthermore, a variable but substantial proportion of patients who are candidates for PPI treatment, such as those with peptic ulcer disease or GERD, have delayed gastric emptying which could be exacerbated by PPI treatment and thus contribute to the frequently observed dyspeptic symptoms seen in these groups of patients (Buckles D. C. et al, 2004, Am J Med Sci, 327, 1-4; Herculano I. R. Jr et al., 2004, Dig Dis Sci, 49, 750-756).

Accordingly, there is a need for agents which modulate and normalize the delay in gastric emptying, particularly the delay in gastric emptying caused by use of PPIs.

It has now been found surprisingly that a HT-4 agonist improves or normalizes delay in gastric emptying, e.g. the delay in gastric emptying caused by the use of PPIs. For example, tegaserod, a well-known HT-4 agonist, has been found to prevent development of delayed gastric emptying caused by omeprazole, a well-known PPI, in healthy male volunteers (see Example).

The terms “5-HT4 agonist” and “PPI” are used herein throughout the patent application as follows:
A “5-HT4 agonist” is an agent that has an affinity for serotonin receptor type 4 and is able to mimic the effects of serotonin by stimulating the physiologic activity at the cell receptor, as e.g. is useful in the treatment of certain gastrointestinal diseases including IBS-C (irritable bowel syndrome with constipation) e.g. in woman, and chronic constipation. Examples are tegaserod, zacopride, pruclopride, mosapride, and norcisapride.
A “PPI” (Proton Pump Inhibitor) is an agent that blocks the transport of hydrogen ions into the stomach and hence is useful in the treatment of gastric hyperacidity, as e.g. observed in ulcer disease or GERD. Examples are omeprazole, esomeprazole, tenatoprazole, (R)-tenatoprazole, (S)-tenatoprazole, rabeprazole, lansoprazole, and pantoprazole.

Accordingly the present invention provides a method for the treatment of delayed gastric emptying, for example caused by PPIs, in a patient in need of such treatment, which comprises administering an effective amount of a 5-HT-4 agonist to the patient.

The invention further provides the use of a 5-HT-4 agonist in the preparation of a medicament for the treatment of delayed gastric emptying, for example caused by PPIs.

Furthermore, the invention provides a pharmaceutical composition for use in the treatment of delayed gastric emptying for example caused by PPIs, comprising a 5-HT4 agonist and suitable excipients.

The uses, pharmaceutical compositions and methods of the present invention represent an improvement to existing therapy of delayed gastric emptying caused by PPIs.

Thus in particular embodiments the invention provides:
a method for the treatment of delayed gastric emptying, for example caused by a PPI, in a patient in need of such treatment which comprises administering an effective amount of a 5-HT4 agonist to the patient;
use of a 5-HT4 agonist in the preparation of a medicament for the treatment of delayed gastric emptying, for example caused by a PPI; and
pharmaceutical compositions for use in the treatment of delayed gastric emptying, for example caused by a PPI, comprising a 5-HT-4 agonist and excipients.

The 5-HT4 agonist used in the present invention are typically those which improve delayed gastric emptying, in particular those which improve delayed gastric emptying caused by PPIs.

Thus, for example, suitable 5-HT4 agonist for use in the invention may include (but are not limited to) the following compounds and pharmaceutically acceptable salts thereof, and any hydrate thereof: tegaserod, zacopride, pruclopride, mosapride, and norcisapride. Other useful 5HT4 agonists include E360, ABT224, VIO134, AT17505 and TD2749.

In one embodiment, the 5-HT₄ agonist for use in the present invention is selected from a compound of formula I:

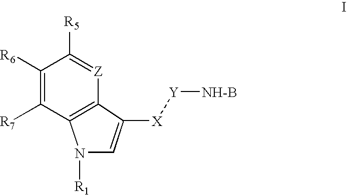

wherein
R₁ is hydrogen; C₁₋₆alkyl; (C₁₋₆alkyl)carbonyl; benzoyl; or phenylC₁₋₄alkyl-carbonyl;
R₅ is hydrogen; halogen; C₁₋₆alkyl; hydroxy; nitro; amino; C₁₋₆alkylamino; C₁₋₁₀alkyl-carbonylamino; C₂₋₆alkoxycarbonyl; SO₂NRₐR_{b} wherein each of Rₐ and R_{b} independently is hydrogen or C₁₋₆alkyl; cyano; or trimethylsilyl; C₁₋₆alkyl substituted by —SO₂—C₁₋₆alkyl, —SO₂NRₐR_{b}, —CONRₐR_{b}, —NH—SO₂—C₁₋₆allcyl, —N(C₁₋₆alkyl)-SO₂—(C₁₋₆allcyl), —NRₐR′_{b} wherein R′_{b} is hydrogen or C₁₋₆alkyl, C₂₋₆alkoxycarbonyl or —PO(C₁₋₄allcyl)₂; carboxy; CONRₐR_{b}; —PO(C₁₋₆alkyl)₂; OCONR_{c}R_{d}, wherein each of R_{c} and R_{d} independently is C₁₋₆alkyl;
R₆ is hydrogen or, when R₅ is OH, R₆ is hydrogen or halogen,
Z is —CR₄═ wherein R₄ is hydrogen, halogen, hydroxy or C₁₋₆alkyl or, when R₅ is hydrogen or hydroxy, Z is also —N═,
R₇ is hydrogen, halogen, C₁₋₆alkyl or C₁₋₆alkoxy,
X—Y is —CR₈═N— or —CH(R₈)—NH— wherein R₈ is hydrogen or C₁₋₆alkyl, and
B is a radical of formula (a) or (b),

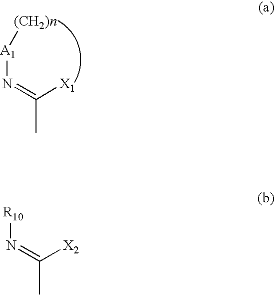

wherein
n is 1 or 2,
A₁ is C═O or CH₂,
X₁ is S; NR₁₁ wherein R₁₁ is hydrogen, (C₁₋₆allcyl)carbonyl, benzoyl or phenylC₁₋₄allcyl-carbonyl; or CR₁₂R₁₃ wherein each of R₁₂ and R₁₃ independently is hydrogen or C₁₋₄alkyl,
R₁₀ is hydrogen; C₁₋₁₂alkyl; C₁₋₆alkyl substituted by hydroxy, aryl, aryloxy, adamantyl, a heterocyclic radical, —NR₁₅—CO—R₁₆ or —NH—SO₂-aryl; C₅₋₇cycloalkyl; adamantyl; (C₁₋₁₀alkyl)carbonyl; benzoyl; phenyl(C₁₋₄allyl)carbonyl; or —CONHR₁₄, wherein
R₁₄ is C₁₋₁₀alkyl or C₅₋₇cycloalkyl,
R₁₅ is hydrogen or C₁₋₄allcyl, and
R₁₆ is C₁₋₆alkyl, C₅₇cycloalkyl, C₅₋₇cycloalkyl-C₁₋₄alkyl, aryl or arylC₁₋₄alkyl, wherever “aryl” appears as is or in the significances “aryloxy”, “—NH—SO₂-aryl” or “aryl(C₁₋₄alkyl)” in the above definition, it is phenyl or phenyl substituted by halogen, C₁₋₄allcyl or C₁₋₆alkoxy; and
wherever “heterocyclic radical” appears in the above definition, it is pyridyl, imidazolyl, benzimidazolyl, pyrrolidinyl, pyrrolidonyl, piperidino, pyrazinyl, perhydroindolyl or a radical of formula (c), (d) or (e)

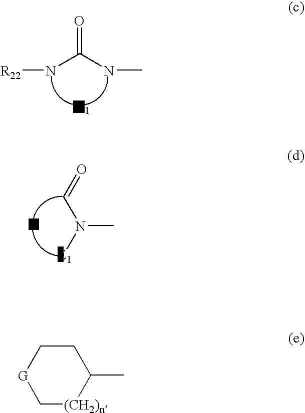

wherein
R₂₂ is hydrogen or C₁₋₄alkyl,
B₁ is —CH₂CH₂—, —COCH₂— or —(CH₂)₃— in which one or two H thereof can by replaced by C₁₋₄alkyl, or 1,2-phenylene,
E is —CH₂—CH₂—, —CH₂N(R₁₇)— or —(CH₂)₃— in which one or two H thereof can be replaced by C₁₋₆alkyl, or 1,2-phenylene,
E₁ is CO or CH₂,
R₁₇ is hydrogen or C₁₋₄alkyl,
G is CO, —CHCOOR₁₈, —CHCOR₁₉, 5,5-dimethyl-1,3-dioxan-2-ylidene or 1,3-dioxolan-2-ylidene, wherein R₁₈ is hydrogen or C₁₋₆alkyl and R₁₉ is C₁₋₆alkyl, and
n′ is 0 or 1, and
X₂ is —SR₂₀ or —NR₃R′₁₀ wherein R₂₀ is C₁₋₆alkyl, R₃ is hydrogen or C₁₋₆alkyl and R′₁₀ has one of the significances given for R₁₀ above, or R₃ and R′₁₀ together with the nitrogen atom to which they are attached form a heterocyclic radical as defined above;
with the proviso that where B is a radical of formula (b), only one of R₁₀ and R′₁₀ can be other than hydrogen and X₂ can be —SR₂₀ only when R₁₀ is hydrogen,
   and a physiologically-hydrolysable and -acceptable ether or ester thereof when R₅ is hydroxy, in free form or in salt form.

Compounds of formula I and their physiologically-hydrolysable and -acceptable ethers or esters are e.g. as disclosed in EP-A1-0 505 322. Suitable pharmaceutically acceptable salts are, e.g., pharmaceutically acceptable acid addition salts, for example such salts as obtained with an inorganic or organic acid, e.g. the hydrochloride, sulfate, acetate, oxalate, maleate and fumarate salts.

By the term “physiologically-hydrolysable and -acceptable ethers or esters” as applied to the compounds of formula I when R₅ is hydroxy, is meant ethers in which R₅ is etherified (e.g. by optionally substituted C₁₋₆alkyl) and esters in which R₅ is esterified and which are hydrolysable under physiological conditions to yield an alcohol or acid which is physiologically acceptable, i.e. which is non-toxic at the desired dosage levels. Specific examples are given in EP-A1-0 505 322.

Preferred compounds of formula I as 5-HT₄ receptor partial agonists are e.g. those wherein R₁ is H, Z is —CH═ and R₅ is OH or C₁₋₆alkoxy.

Further examples of 5-HT₄ receptor partial agonists include e.g. RS 67333 (1-(4-amino-5-chloro-2-methoxyphenyl)-3-[1-butyl-4-piperidinyl]-1-propanone), or RS 67506 (1-(4-amino-5-chloro-2-methoxyphenyl)-3-[1-methylsulphonylamino)ethyl-4-piperidinyl]-1-propanone).

A particularly preferred compound of formula I is the compound of formula

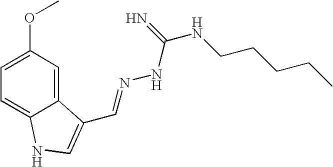

in free form or in pharmaceutically acceptable salt form. This compound has the chemical name of 3-(5-methoxy-1H-indol-3-yl-methylene)-N-pentylcarbazimidamide, and is also known as tegaserod. It is disclosed as being a 5-HT₄ receptor partial agonist. It may also exist in form of tautomers

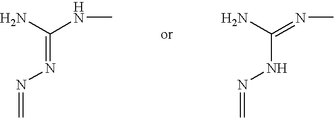

which are included in the present invention. A preferred salt form is the hydrogen maleate. A preferred crystalline form is as described in WO 2005/014544.

All the 5-HT4 agonists mentioned above are well known from the literature. This includes their manufacture. For example, tegaserod is prepared as described e.g. in U.S. Pat. No. 5,510,353.

The 5-HT4 agonists (hereinafter referred to as the Agents of the Invention) may be used in the form of an isomer or of a mixture of isomers where appropriate, typically as optical isomers such as enantiomers or diastereoisomers or geometric isomers, typically cis-trans isomers. The optical isomers are obtained in the form of the pure antipodes and/or as racemates.

The Agents of the Invention can also be used in any salt form or in the form of their hydrates or include other solvents used for their crystallisation.

The Agents of the Invention are preferably used in the form of pharmaceutical compositions that contain a therapeutically effective amount of active ingredient optionally together with or in admixture with inorganic or organic, solid or liquid, pharmaceutically acceptable carriers which are suitable for administration.

The pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

Preferably, the pharmaceutical compositions are adapted to oral or parenteral (especially intravenous, intra-arterial or transdermal) administration. Intravenous and oral, first and foremost intravenous, administration is considered to be of particular importance.

The particular mode of administration and the dosage may be selected by the attending physician taking into account the particulars of the patient, especially age, weight, life style, activity level. Most preferably, however, the Agents of the Invention is administered orally.

The dosage of the Agents of the Invention may depend on various factors, such as effectiveness and duration of action of the active ingredient, mode of administration, warm-blooded species, and/or sex, age, weight and individual condition of the warm-blooded animal.

The dose mentioned above may be administered as a single dose or in several partial doses—may be repeated, for example once, twice or trice daily. In other words, the pharmaceutical compositions may be administered in regimens ranging from continuous daily therapy to intermittent cyclical therapy.

Preferably, tegaserod hydrogen maleate, one of the Agents of the Invention, is administered in doses which are in the same order of magnitude as those used for the treatment of Irritable Bowel Syndrome diarrhea predominant (IBS-C), e.g. 6 mg trice daily.

The following Example illustrates the invention described hereinbefore.

### EXAMPLE

A Randomized, Double-Blind, Placebo-Controlled Study to Evaluate the Effect of Tegaserod on Delayed Gastric Emptying Associated with Omeprazole in Healthy Male Volunteers

Methodology: This is a single center, double-blind, randomized, parallel group trial using a placebo control or tegaserod 6 mg t.i.d. in combination with omeprazole 20 mg b.i.d. Gastric emptying is assessed using a standardized method that has been previously validated and found to be sensitive and specific. After an overnight fast, the subjects are asked to ingest a standardized ⁹⁹Tc radiolabeled meal (250 ml EggBeater, the equivalent of two eggs, two slices of white bread, 30 cc of strawberry jam and 120 ml of water). The level of radioactivity in gastric contents is measured by external scintigraphy immediately following completion of the meal and then at 60, 120 and 240 minutes after the end of the meal.

Number of subjects: 40 healthy male volunteers (20 per group)

Investigational drug: Tegaserod 6 mg tablets (supplied by Novartis Canada, but are normal 6 mg Zelnorm tablet obtainable as prescription) for oral administration. Each dose consists of a tablet to be taken with a glass of water within 15-30 minutes before mealtime three times daily. Open label component: omeprazole 20 mg tablets (purchased in local pharmacy) to be taken with morning and evening doses of tegaserod or placebo.

Reference therapy: Matching tegaserod 6 mg placebo tablets for oral administration. Each dose consisted of a tablet to be taken with a glass of water within 15-30 minutes before mealtime three times a day. Open label component: omeprazole 20 mg tablets to be taken with morning and evening doses of tegaserod or placebo.

Duration of treatment: Subjects are expected to take their study drugs for 14 consecutive days.

Criteria for Evaluation:

Efficacy: The primary efficacy parameter is time to half emptying (T_{1/2}) of gastric contents. The secondary efficacy endpoints were the calculated lag phase, and percent of gastric contents remaining in the stomach at 60, 120 and 240 minutes after ingestion of a standard meal.

Statistical methods: Paired T-test and Sign Test are used to compare changes in gastric emptying parameters from baseline in two treatment groups.

Results:

Efficacy: Monotherapy with omeprazole 20 mg b.i.d. for 14 days delayed gastric emptying as measured by T_{1/2} (p<0.003) and by percent of fond remaining in the stomach at 60 minutes (p<0.002) and 120 minutes post-test meal (p<0.04, all by paired T-test). When omeprazole is administered in combination with tegaserod 6 mg t.i.d., gastric emptying at all the studied time points is not statistically different from the respective baseline values.

Conclusions: Addition of tegaserod to omeprazole prevented development of delayed gastric emptying caused by omeprazole monotherapy in healthy male volunteers.

1-5. (canceled)

6. A method of preventing or treating PPI-induced delayed gastric emptying, comprising administering to a subject in need thereof an effective amount of a 5-HT4 agonist.

7. The method of claim 6, wherein the PPI is selected from omeprazole, esomeprazole, tenatoprazole, (R)-tenatoprazole, (S)-tenatoprazole, rabeprazole, (R)-rabeprazole, (S)-rabeprazole, lanzoprazole, (R)-lanzoprazole, (S)-lanzoprazole, pantoprazole, (R)-pantoprazole and (S)-pantoprazole and pharmaceutically acceptable salts thereof.

8. The method of claim 6, wherein the 5-HT4 agonist is tegaserod, zacopride, pruclopride, mosapride, norcisapride or pharmaceutically acceptable salts thereof.

9. The method of claim 8, wherein the 5-HT4 agonist is tegaserod hydrogen maleate.
